(19) United States
(12) Patent Application Publication
(10) Pub. No.: US 2005/0032218 A1
(43) Pub. Date: Feb. 10, 2005

(54) Method to manufacture a cell preparation and such manufactured cell preparations

(76) Inventor: Joerg C. Gerlach, Pittsburg, PA (US)

Correspondence Address: Joerg C. Gerlach MD, PhD, 3613 Butler St, Pittsburgh, PA, 15201, US

(21) Appl. No.: 10/866,273

(22) Filed: Jun. 12, 2004

(30) Foreign Application Priority Data
Jun. 13, 2003 (DE) 103 26 750.6

(51) Int. Cl. C12N 5/08; C12N 15/85

(52) U.S. Cl. 435/455; 435/366

(57) ABSTRACT

This invention pertains to a method for the manufacturing of a cell preparation as well as such manufactured cell preparations. As a special form of such a cell preparation it also pertains to methods of generating organ/cell transplants and such a generated organ/cell transplants as well as subsequent applications. Such cell preparations are of interest for extracorporeal support systems, such as liver support, or transplants/cell implants that are transferred into organs, for instance the liver, or are placed in various other areas of an organism.

### DESCRIPTION

This invention pertains to a method for the manufacturing of a cell preparation as well as such manufactured cell preparations. As a special form of such a cell preparation it also pertains to methods of generating organ/cell transplants and such a generated organ/cell transplants as well as subsequent applications.

Such cell preparations are of interest for extracorporeal support systems, such as liver support, or transplants/cell implants that are transferred into organs, for instance the liver, or are placed in various other areas of an organism.

It is known that for instance the liver possesses a remarkable ability to regenerate and proliferate. The source of proliferating liver cells however has not been conclusively characterized to date. In particular, uniform criteria do not exist for the characterization of organ stem cells, respectively progenitor cells. As a result, in state-of-the-art technology, various and partly conflicting markers are used/published to characterize the stem cell populations of different organs. The main effort is to isolate only those stem cells, e.g. liver progenitor cells, from the remaining organ cells that have been appropriately marked. This can be achieved by using surface molecules, morphological and immunehistochemical markers that are defined in various ways as described above or by using different cell sizes and/or densities. In addition, the heterogenic terminology that is used to mark, for instance non-differentiated liver cells that have the ability to proliferate and differentiate, indicates that the situation is still unclear in regards to organ stem cells that have the ability to proliferate and differentiate. For example terms like “hepatocyte precursors”, “liver progenitor cells”, “liver stem cells”, “small hepatocytes”, “liver parenchyma cells with cloning growth ability”, or “immature hepatocytes” are used.

In front of the backdrop of experiments to gain stem cells from adult tissue, the task of the invention on hand is, to develop a method to manufacture cell preparations with which proliferating and differentiating stem cells can be isolated from any tissue or organ. Such manufactured cell preparations lie within the potential of the invention on hand as well as therewith generated organ transplants/cell implants and such.

This task has been achieved via a method according to claim 1 or 6, the cell preparation according to claim 52, including further depending methods. Advantageous developments of the respective methods, cell preparations and organ transplants/cell implants are described in the respective depending claims.

The invention at hand separates itself from current known technology through a completely different approach to gain stem cell preparations, respectively cell preparations, enriched with cells able to proliferate and/or differentiate. The approach of the invention at hand is to completely forego the elaborate marking of selected cells and the subsequent separation/fractioning process, but use a non-fractioned cell combination that contains all cells, which are for instance present in the original tissue, e.g. in the liver in its physiological conditions.

Therefore the originating material contains all differentiated and non-differentiated cells, including those cells able to proliferate and differentiate that are contained in the original tissue, independently of their molecular surface pattern, size, density, morphological or immunehistochemical profile. It is critical for the invention that the unfractioned cell mixture is exposed to a selective and/or partial damage for instance through ischemia, hypoxia, exposure to temperature, chemical noxa, or mechanical influences and such. This process causes the mature highly differentiated cells to become more selectively damaged then the undifferentiated cells, and/or the undifferentiated cells better recover from the damage in comparison to the highly differentiated cells. Therefore the principle of the invention at hand is to utilize the higher resistance of undifferentiated cells towards environmental factors when compared to their differentiated offspring.

The damage can occur before, at the same time, or after the generation of the unfractionated cell mixture. This means that the damage can occur, for example, in the originating organ or originating tissue, after the dissociation of the cell formation, or during any one of these processes.

Another principle of the invention at hand is to offer culture conditions corresponding to those in a desired tissue to the unfractionated cell mixture after extraction of selected cell fractions, or to a stem cell culture.

The desired tissue does not have to correspond with the original tissue or organ. It is for instance possible to attain an unfractionated cell mixture from live tissue, as described above, yet aim to attain differentiated cells of another tissue from liver stem cells that were selectively attained as described above. A further example is the application of bone marrow ells/peripheral blood stem cells for the generation of liver cells. This can also be accomplished through an already existing stem cell culture by offering cell growth-, cell division-, and/or cell differentiation conditions that correspond with the desired cell tissue. This occurs, for instance, by cultivating cell mixtures or cell cultures in bioreactors, as described in the EP 93 11 40 76.8 (EP 0 590 341) (Gerlach J. C.) U.S. patent Ser. No. 08/117,429: 1993, or, along with the claim at hand, as described in a simultaneously submitted claim with the titles “bioreactor from one block” and “organ cast”, submitted by the same claimant. The disclosure of this claim with respect to the design and construction is explicitly included in the registration at hand.

Thus the regeneration of the cells under the stimulation of reproduction, respectively the differentiation of such enriched stem cells in vitro is caused through the simulation of natural regeneration processes.

The deteriorating, pre-damaged, differentiated cells as well as the stem cells themselves, release factors that support/enhance cell proliferation/differentiation processes. This can also be achieved by adding autologous, non-parenchymal (mesenchymal) cells of the same organ or another organ or tissue from the same donor. Therewith, natural chemical signals are created that support the cell proliferation, respectively cell differentiation, into the specific cell types of the respective host tissue. Additionally, homologous or heterologous non-parenchyma cells (mesenchymal cells) of the same organ or other organs/tissues of a different donor can be added.

Such an organ specific micro-environment in which the enriched/isolated cells are located, causes the enriched/isolated cells to regenerate and specifically differentiate into differentiated cells that correspond, in function and structure, to cells of other organs or tissues. By selecting specific co-culture cells the desired target organ/target tissue can be determined. To facilitate the regeneration, proliferation, and differentiation of the isolated cells, the cells of the target tissue can be selectively damaged. As a result, the invented method allows for a simple and effective way to attain stem cells from adult or fetal tissue, which in turn can be maintained under target organ/target tissue specific conditions. Therewith, for the first time it is possible to, easily and simply, generate a stem cell pool, and to manufacture differentiated cells from selected target tissue/organs.

Subsequently to the damage, cells with a certain degree of damage can be separated; meaning cells that have not at all been damaged or have been less damaged, i.e. mostly undifferentiated stem cells, or heavily damaged cells, i.e. mostly differentiated cells.

Advantageously, the invention can be used to generate a cell implant as well as a modified transplant. Hereunto a cell preparation, as described above, is inserted into an already existing organ transplant, as described above, for instance by means of an in-vitro perfusion system. The transplant functions as a biological scaffold. Subsequently, the cells in the transplant are being damaged selectively or partially. By creating a selective advantage of survival for the imported cells, e.g. through noxa or by importing appropriate immune competent cells, and subsequent proliferation/differentiation of the imported cells in the organ transplant, an organ transplant inside a perfusion system is created that consists partially of imported cells and partially of cells from the biological scaffold (modified biological scaffold). Such transplants have the advantage to possibly avoid rejection, inasmuch as autologous cells were imported into the homologous original transplant. Once the advantage for survival for the imported autologous cells has been gradually improved, respectively repeated, the imported cells and their offspring will, in the course of time, replace the biological scaffold so that an organ is gradually generated that consists primarily of imported cells (in vivo tissue engineering). The invention can also serve to manufacture an organ transplant, which is thereby characterized that it contains cells, derived from the original transplant as well as imported cells, which are distributed in at least one supply area of an artery in one cell organization.

Thereby, the culture of the cell preparation or the transplant can advantageously occur in bioreactors. A particularly effective arrangement is described in the EP 059 034 A2. Thereabouts described module, for the culture and utilization of metabolic activity and for the maintenance of microorganisms, consists of an outer container with at least three independent membrane systems arranged therein. At least two of these membrane systems are developed as hollow fiber membrane systems and are arranged in the interior of the module. The hollow fiber membranes form a tightly packed spatial network. The microorganisms are thereby attached to the hollow fiber membranes and/or the hollow spaces inside the network.

One independent hollow fiber membrane system serves for the media inflow. A second independent hollow fiber membrane system designed to supply the microorganisms with for instance oxygen and to remove CO₂. The media outflow is secured through a third independent membrane system.

Each individual hollow fiber membrane system consists of numerous individual hollow fiber membranes, whereby each hollow fiber of a system communicates with at least one inflow, respectively one inflow and one outflow. This guaranties that the hollow fibers of a particular system can be simultaneously supplied with media through the inflow.

The independent hollow fiber membrane systems form a tightly packed spatial network inside the module in such a way that, at almost any location of the network, the microorganisms have almost identical conditions for the substrate supply. Thereby, the conditions in the physiological organs with their own arteries and veins, e.g. the liver with hepatocytes arranged in the lobuli, are largely simulated. Via the independent arrangement of the various membrane systems the module offers the advantage of decentralized transportation of for instance nutrients, synthesis products and gases to/from numerous microorganisms independently of their position inside the module, as it is the case in the cell environment of the natural organ.

The media outflow is thereby ensured through the third independent membrane system. This membrane system can consist of hollow fiber membranes, exchangeable flat membranes, or exchangeable capillary membranes. It is critical that also the third membrane system is independent from the other two hollow fiber membrane systems.

One arrangement suggests that the tightly packed network in the interior is formed by three independent hollow fiber membrane systems, in which case all independent membrane systems are hollow fiber membranes arranged in the interior. One independent hollow fiber membrane system serves for media inflow, one serves for media outflow, and a third serves for additional supply with for instance oxygen. Then the tightly packed network consists of these three independent systems.

The tightly packed network can be arranged in various ways as long as it is guaranteed that the microorganisms in the interior have an identical substrate supply. The spatial tightly packed network can for instance consist of tightly packed layers, whereby layers of independent systems alternate. The first layer consisting of individual hollow fiber membranes is arranged horizontally. The second layer, also consisting of individual hollow fiber membranes, is also arranged in the same plain but rotated versus the first layer for instance at a 90 degree angle. Theses layers alternate and form a tight package. The third independent hollow fiber membrane system, also consisting of individual layers of hollow fiber membranes, crosses the first two independent layers for instance vertically from top to bottom, and therewith interweaves the two other independent layers.

A further arrangement is designed to alternately layer three independent hollow fiber membrane systems in one plain but each rotated at 60 degrees.

This tightly packed network is arranged in the interior of the module. Because each individual system communicates with at least one inflow, respectively one inflow and one outflow, an even distribution of inflowing media inside the module as well as an even oxygen distribution are guaranteed. Via the third independent system for the media outflow, media can be consistently removed from anywhere in the module.

In a further arrangement an additional independent membrane system for the media outflow is used in addition to the three hollow fiber membrane systems in the interior. For that purpose, exchangeable flat membranes or exchangeable capillary membranes can be attached on the outer casing. This arrangement assures the trouble-free outflow of media even over extended periods of time.

In a further arrangement, the tightly packed network is formed by two independent hollow fiber membrane systems whereby one serves for the media inflow, the second serves for oxygen supply, and a third independent membrane system, in form of exchangeable flat- or capillary membranes, serves for the media outflow.

The tightly packed network in the interior that is formed through two hollow fiber membrane systems, is constructed analogous to those afore described.

Polypropylene, polyamid, polysulphone, cellulose, or silicon is preferably used for hollow fiber membranes. The selection of hollow fiber membranes depends on the molecules responsible for substance/mass exchange. However, all state of technology hollow fiber membranes for substance/mass exchange can be applied.

Should three independent hollow fiber membrane systems that form a tightly packed network in the interior be used, a fluid impermeable capillary system made of, for example, stainless steel or glass can be used. This can serve to temper the module's interior. Likewise, it allows for an even cooling of the module's interior and the infused microorganisms below −20 degrees Celsius. In another arrangement all other hollow fiber systems can be used to temper/cool below the freezing point.

In a further arrangement the outer casing is formed through a casting material, whereby an access into the volume of the capillaries is always guaranteed.

In a further arrangement the module exhibits various accesses. One access serves to infuse the microorganisms into the module. Additional accesses serve for pressure-, pH— and temperature measurements in the interior of the module.

This bioreactor shows excellent results in regards to substrate supply and —removal of the microorganisms. A further module is known from the application submitted by the same inventors on the same day as this application with the title “Module for the culture and utilization of metabolic processes and/or for the maintenance of microorganisms”. This module consists of a body that is arranged in a water tight and sterile container, whereby the body exhibits pores that can communicate with each other. Simultaneously this body exhibits at least one channel like hollow pathway system whose individual hollow pathways penetrate the body while crossing and/or overlaying each other. Because the body, arranged inside the container, consists of porous material whose pores can communicate with each other, a connection between the pores is secured via the independent channel like hollow pathway systems. The microorganisms inside this module, especially the cells, are firmly fixed inside the pores of this porous body without completely filling them out. Via the independent channel like hollow pathway systems, arranged inside the body, an even substrate supply and removal with low substance gradients can occur for the microorganisms, especially for the cells, located inside the pores.

Therewith, this module copies the cell supply as it occurs in the natural organs. Thus this module represents a bioreactor that permits an optimal substrate supply—and removal of a relatively large amount of microorganisms over extended periods of time anywhere inside the bioreactor.

A channel like hollow pathway system is preferably arranged in such a way that it consists of parallel running channels arranged in one plain. A channel like hollow pathway system constructed of several such plains arranged at a pre-determined distance on top of each other is preferred. The distance of the individual channels of a hollow pathway system in one plain and in between the individual plains can be around 1-5 mm. The diameter of the individual channels is preferably 0.1-2 mm.

The body of the module can contain at least two such hollow pathway systems cross each other and/or overlay each other. Therewith a substance exchange, across both hollow pathway systems, respectively between both hollow pathway systems, is possible in counter current process with relatively high capacity and simultaneously low substance gradient.

In a preferred arrangement the hollow pathway systems cross each other. As a result, one hollow pathway system, preferably consisting of several overlaying plains, penetrates the body in one direction and a second hollow pathway system penetrates the body at a 90-degree angle from the other direction.

When the plains are arranged on top of each other in the defined distance, a substrate supply and—removal of the microorganisms inside the pores of the porous body is possible anywhere inside the body. This module also contains all other arrangements in regards to the geometrical arrangement of the hollow pathway systems to each other, as long as a nearly identical substrate supply and—removal from anywhere inside the body is guaranteed. The two hollow pathway systems can cross each other in a predetermined angle. They can also be arranged on top of each other whereby the counter current principle can be utilized at an optimal.

If the module exhibits a third hollow pathway system, it is also formed from parallel-arranged hollow pathways in one plain. This hollow pathway system penetrates the body for instance vertically form top to bottom and interweaves the first two independent hollow pathway systems whereby additional decentralized functions like oxygenation can be integrated. With this third independent hollow pathway system, the module naturally contains all geometrical arrangements as long as an identical supply and removal of the microorganisms/cells is guaranteed anywhere inside the body.

Analogous, a fourth or more hollow pathway systems can be integrated, which would facilitate additional functions like cell drainage for cell production.

In this module, the first independent hollow pathway system can serve for instance for the outlet of the media. The second independent hollow pathway system serves for the supply of the microorganisms for instance with oxygen, respectively the removal of CO₂. This can also occur by threading gas permeable oxygenation fibers from blood oxygenators into the hollow pathway system. The media outflow is then secured by the second independent hollow pathway system. Alternatively, the first and second hollow pathway system can be operated in counter current flow whereby the perfusion of the cells is achieved via increasing pressure gradients between both systems.

Afore closely described channel like hollow pathway systems infuse the porous body of described module. The dimensions of the pores of the porous body are larger then a cultivated cell. Therefore, the pores of the porous body exhibit a diameter of 50-1000 micrometer. The significance of this body is that the pores are connected through pore wall openings to facilitate the optimal in—and outflow of media across many pores. The pores are connected with each other through openings of about 50-300 micrometer in diameter. This arrangement guarantees that the inflowing media can reach every part of the porous body via the independent hollow pathway system and that the removal of outflowing media from anywhere in the hollow structure via the pores and their connections to the channels of the hollow pathway system is secured. Therefore, the porous body can be referred to as a foam-/sponge structure.

The porous body inside the container can exhibit any geometrical shape. Importantly, the porous body has to exhibit a volume that can accommodate enough cells/microorganisms depending on the application. Therefore the porous body has a volume of preferably 0.5 ml to 5 l.

The geometrical form is optional. However, a block shape is preferred because it simplifies the insertion of hollow pathway systems from one side to the other and a second from an additional side to another. Cuboids or other rectangular hollow block shapes are preferred. A more complex outer casing is only necessary when the number of hollow pathway systems exceed three.

The porous, block shaped body can be manufactured in one piece or it can consist of several overlaying disc/slide shaped individual layers, which are held together by the container.

In regards to afore mentioned disc/slide shaped alternative, it is advantageous if at least one layer of the disc/slide shaped, individual layers is fitted with channel like rides. These channel shaped ridges are drilled into the surface in such a way that they form a channel like hollow pathway system in connection with the following individual layer.

Therefore the ridges are shaped as semi channels so that in connection with the following individual layer a full channel develops. The advantage of this arrangement is that it is procedurally easy to drill ridges into the individual layers. It is advantageous if the individual discs/slides exhibit, on their front wall, the second channel like hollow pathway system in form of infused channels. Therewith, via the development of these individual layers and their interconnections, a porous body with two independent hollow pathway systems is created. One hollow pathway system is formed by the ridges in the surface of the individual layers, whereby the second hollow pathway system is created by the channel-like hollow pathways infused into the individual disc/slide.

A third hollow pathway system can result from drilling into the remaining plain/surface of the discs/slides.

The porous body, as described afore, is arranged inside a container. The arrangement of the water tight/sterile container and porous body is developed in such a way that the channel-like hollow pathways of a system meet in at least one inlet and outlet. The inlet/outlet is developed in such a way that they pass through the container, and thereby secure the supply and waste removal of the hollow body from outside the container. In general there are two constructions possible. One is that the inlet and outlet is part of the container itself and the connections are created through the arrangement of the body inside the container or the inlet and outlet, and be connected with the porous body in which case it is surrounded by a water tight and sterile container.

The container can be developed in form of a casing or a foil. A container in form of a casing is preferred especially the use of an injection molding casing. All state of the art materials for injection molding casings, e.g. polycarbonate are useable. It is advantageous that the container and the connections can be generated from re-absorbable/biodegradable material in order to use the module as implant.

The porous material preferably consists of sintered ceramic powder. The use of hydroxyapatite is particularly favored. Hydroxyapatite is a calcium phosphate, which is a ceramic substance with various parts of calcium and phosphor. Hydroxyapatite is a compound that exists in nature but can also be synthetically generated.

The medical use of hydroxyapatite as bone replacement substance is already known. The motivation for the clinical use of hydroxyapatite is to apply a material of similar composition as the mineral phase of bone marrow. Hydroxyapatite exists as a natural component in the mineral part of bone marrow with 60-70%. Hydroxyapatite is generated for instance via precipitation method from a watery fluid in a calcium nitrate solution at a basic pH by adding for instance ammonium phosphate. Fusion of the powder particles can occur via sintering process at 1000 to 2000 degrees Celsius. For the manufacturing of porous solid bodies from hydroxyapatite, e.g. open pore, foam like structures, hydroxyapatite is mixed with organic additives, which are later burnt out under high temperatures (Wintermantel et al.: Biokompatibler Werkstoff und Bauweise: Implantate fuer Medizin und Umwelt Berlin/Springer 1998: 256-257)

A further bioreactor by the same inventors is described simultaneously to the registration at hand with the title “Bioreactor in form of an organ copy, process of its manufacturing and application for the culturing, differentiation, maintenance and/or utilization of cells”. In this case the bioreactor exhibits a container in which a porous body is arranged whose pores are in communication with each other. Additionally, at least two independent channel like hollow pathway systems are arranged inside this body that cross each other and/or overlay each other and infuse/cross through the body.

In this case, cells settle inside the body's pores and are immobilized in respect to their position.

Thereby a bioreactor in form of an organ copy is provided. The hollow structures of the bioreactor allow for the maintenance of a larger, highly dense cell mass. The fluid exchange to/from the cells occurs decentralized via blood plasma or media avoiding significant substrate gradients. Hollow structures pertain to supplying vessels (arteries), discharging vessels (veins), as well as other organ typical vessels for example the liver portal veins in the liver, liver-bile duct, and the canals of Hering with the liver stem cells.

Significant in this bioreactor is that its immunological inactive porous body exhibits pores that can communicate with each other. The pores exhibit a size that exceeds the size of the cells of the respective organ. Therefore the pore diameter lies between 50-1000 micrometers. The pores are interconnected through pore wall openings. These openings are preferably channel like and about 50-300 micrometer in size. This arrangement guarantees that the pores intercommunicate, via the pore openings, with the hollow structures of the organ copy. The afore mentioned structure of the porous body can also be referred to as a foam-/sponge like structure.

It is critical that the bioreactor is formed from an immunological inactive, perfuseable foam-/sponge like structure in which the cells are settled inside the hollow spaces and the pores of the foam structure communicate with each other.

Therefore, the pores facilitate media perfusion, cell infusion, cell migration as well as substrate exchange. Hereby, a bioreactor has been developed which is significantly improved, in comparison to known state of technology bioreactors, with respect to its metabolic exchange structures, efficiency, and features.

This bioreactor describes a device that facilitates the organ typical re-organization of biological cells. The characteristic of this invention is that the specific hollow structures for the maintenance of the cells inside the body are arranged in the same way as provided in nature.

All, so far known state of technology materials generating open pore structures that lead to foam-/sponge like structures according to the invention at hand are suited, for instance ceramics. Particularly suited is hydroxyapatite. Hydroxyapatite is already well known in medicine and analyzed and is therefore particularly suited for this application. Hydroxyapatite exists in form of a powder and can be frothed to the desired foam-/sponge structure by adding pore-building additives and subsequently sintered.

This bioreactor is preferably arranged inside a water- and microorganism tight and germ free container.

Suitable are foils and adequately dimensioned casings. In this case connections are provided that are in contact with at least one hollow structure of the organ casting to ensure an adequate supply—and removal environment inside the bioreactor. With respect to the arrangement of the connections it is certainly possible to combine several inlets and/or outlets of an organ casting to one single inlet and/or outlet. Such solutions for bioreactor are already known from WO 00/75275 (Mac Donald, USA) and EP 1 185 612 (Mac Donald, USA).

Another advantage of this bioreactor is that the container and the connections can be generated from absorbable/biodegradable material, which allows the use of the bioreactor as implant.

With respect to their disclosure contents afore described three registrations are completely incorporated into the registration at hand in regards to the arrangement of the modules/bioreactors, because such bioreactors can also be utilized as bioreactors in the invention at hand.

The utilization of the invented application, especially the cell preparations are described in the patent claims however not representing a concluded list.

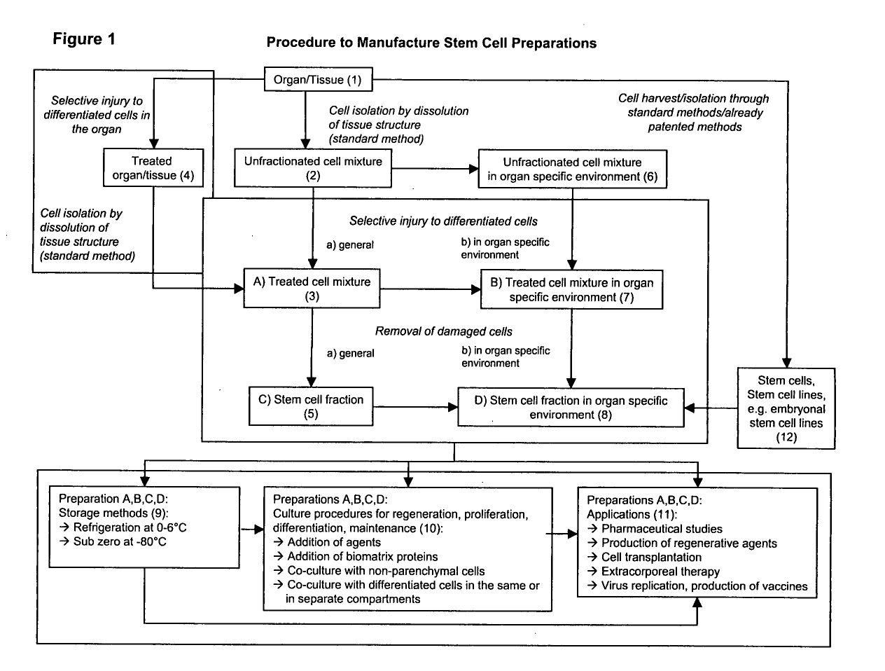

FIG. 1 describes various possibilities of implementation of this invention. Beginning with an organ, respectively tissue 1, the cell formation is dissociated. This can occur, for instance, through enzymatic or mechanical dissociation, or a combination of such processes.

The result is an unfractionated cell mixture 2, in which the fully differentiated cells are subsequently, selectively and/or partially, more severely damaged then the undifferentiated cells. This process results in a treated cell mixture 3, from which the damaged cells are removed to result in a stem cell fraction 5. This stem cell fraction can then be transferred into an organ specific environment 8, for instance be mixed with a cell mixture, which was extracted from a target tissue, or be co-cultured with such a cell mixture by avoiding cell-cell contacts via a membrane but permitting media exchange through the membrane.

Alternatively, the unfractionated cell mixture 2 can be inserted into a bioreactor and thus form an unfractionated cell mixture 6 in an organ specific environment, e.g. in a specific reactor structure. Or the cell mixture can be placed directly into an organ specific environment, for instance by mixing it with a cell mixture from target tissue. Subsequently, the cell mixture can be damaged creating a treated cell mixture 7 in an organ specific environment. The perishing cells will release signals (for instance chemical signals), which will result in the proliferation and/or differentiation of the less damaged or not at all damaged stem cells.

As another alternative, the initial organ/initial tissue 1 can be directly and selectively damaged. Subsequently, the damaged organ/tissue 4 can be dissolved, providing a treated cell mixture containing selectively damaged and undamaged cells.

A further alternative is to attain stem cells/stem cell lines via conventional state of technology processes from a host organ/host tissue 1, e.g. liver stem cells. An embryonal stem cell line 12 can also be directly generated. The stem cells will then be imported into an organ specific environment and for instance be mixed with a cell mixture from a target tissue or target organ, and/or imported into a bioreactor with organ typical-/organ specific structure. As a result a stem cell fraction 8 in an organ specific environment can be generated. For the creation of an organ specific environment factors like cell biological-, biochemical-, chemical-, physical-, and/or structural components are of significance.

Starting with cell mixture 3, stem cell fraction 5, stem cell fraction 8 in an organ specific environment, the treated cell mixture 7 in an organ specific environment, or the treated organ/tissue 4, additional procedures can now follow. These are described in FIG. 1, reference marker 9 through 11. The individual cell preparations can be stored at 0 to 6 degrees Celsius (cool storage) or at −80 degree Celsius (deep-freeze storage) before further treated (see 10 & 11). These cell preparations can also be directly cultivated without prior storage, to regenerate, proliferate, differentiate, or just maintain the concentrated stem cells within, for instance as stem cell pool as stem cell pool.

Active ingredients or matrix proteins can be added. The cell preparations can be maintained in co-culture with non-parenchyma cells and/or in co-culture with differentiated cells. Thereby, it is possible to cultivate the cell preparations and the co-cultured cells in separate compartments for instance in a bioreactor, whereby however, a signal transport across the compartment barrier has to be ensured. This can be accomplished via a barrier membrane that is not permeable for cells but permeable for mediators.

Alternatively, the cell preparations, as described above, can be used directly after storage or after further culture processes according to reference marker 10, for the generation of vaccines, virus proliferation, drug studies, cell transplantation, in-vitro tissue engineering, in-vivo tissue engineering, extracorporeal therapy procedures, and for organ/tissue regeneration through substances generated by the cell culture. Thus, the preparations can also be used for the generation of active agents.

Following are examples for the manufacturing of liver cell preparations through selectively damaging the differentiated liver cells in an unfractionated cell mixture.

FIG. 1 above described overview with respect to the procedures for the manufacturing of stem cell preparations and

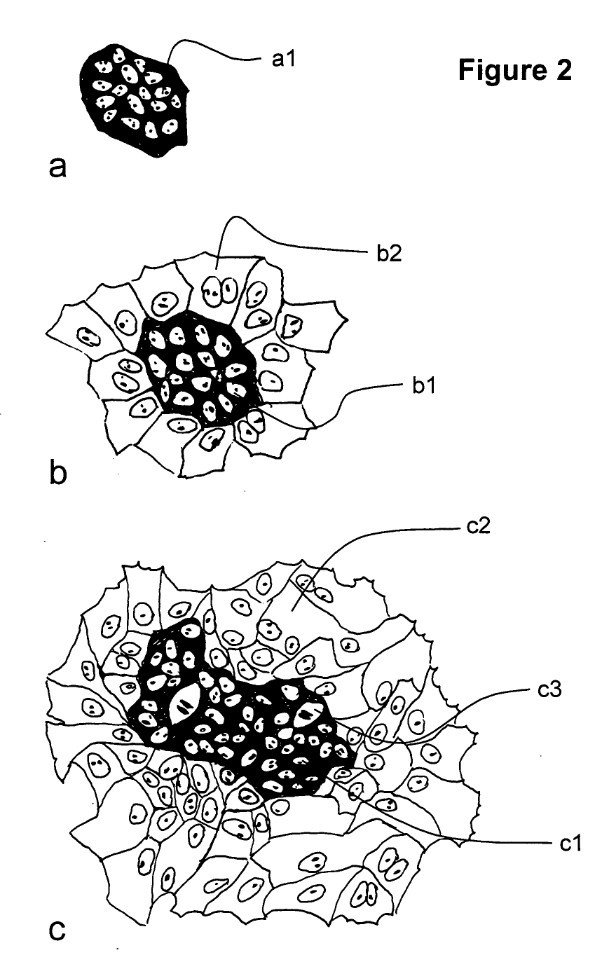

FIG. 2 an illustration of liver stem cell cultures

The principle of the following described methods for the extraction of liver stem cells from human liver tissue is to first achieve damage to differentiated liver cells through long-term hypoxia/anoxia. Through subsequent aggressive protease incubation, the release of the cells from the tissue organization, as well additional damage to the differentiated liver cell is achieved, which conclude in the destruction of these cells. Based on their greater robustness, the undifferentiated liver cells are not or only slightly damaged, which subsequently allows for the cultivation, proliferation and/or differentiation of these undifferentiated liver cells. Regeneration can now occur inside the bioreactors. Selective damage can also be inflicted upon the cells, individually or in combination, through temperature changes (hypothermia, hyperthermia), chemical noxe, mechanical stress, pH alterations, hypotonic conditions, or other damaging factors.

### EXAMPLE 1

### Extraction of Liver Stem Cells from Human Liver Tissue after Partial Resections

For the extraction of liver stem cells from human liver tissue after partial resections, tissue from morphologically intact border areas of the sections was used. After resection, the tissue (4.7-46.4 g) was imported under sterile conditions into a vessel of synthetic material with 20-100 ml (depending on the size of the tissue sections) Williams' Medium E (Williams G M, Weisburger E K, Weisberger J H. Exp Cell Res 1971; 69, 106) with the following additives: 10% fetal calf serum (FCS), 15 mmol/l Hydroxyethylpiperazinethansulphane acid (HEPES), 2 mmol/l L-glutamine, 100.00 IE/l Penicilline, 100.000 microgramms/l Streptomycin and 2.5 mg/l Amphotericin B, and incubated, under exclusion of oxygen, at 4 degrees Celsius for 42-72 hours.

Subsequently, the tissue sections were cut into pieces of 1-2 mm³. To dissociate the tissue and to damage differentiated liver cells, the liver pieces were placed in an enzyme solution that contained 01% collagenase type IV (clostridiopeptidase A; collagen digestive activity: 478 units/g, FALGPA-hydrolytic activity: 2.5 units/g) and 0.1% pronase E (activity: 4.000.000 PU-units/g) in Dulbecco's phosphate buffered saline (PBS) (Dulbecco R., Vogt M. J Exp Med 1954; 99: 167) without the addition of calcium and magnesium (2 ml enzyme solution/g of liver). The enzyme solution with the liver pieces was incubated in a water bath at 37 degrees Celsius and repeatedly swiveled over 60 minutes. Subsequently, the solution was briefly shaken up. After the tissue remains had settled, the supernatant was removed and centrifuged at 600 rotations/minute (Rpm) over 6 minutes at 4 degrees Celsius in order to separate the damaged cells. The cell pellets containing the less sensitive cells were suspended in a hypertonic solution, consisting of 10 mmol/l KHCO₃, 155 mmol/l NH₄CL, 0.13 mmol/l ethylendiethyltetraacetate (EDTA) in distilled water with a pH 7.5 (1 ml/liver). The cell suspensions were incubated in this solution for 5 min at 4 degrees Celsius to destroy the erythrocytes contained in the suspension. Subsequently, the suspensions were centrifuged again at 600 RPM for 6 min at 4 degrees Celsius to separate the damaged erythrocytes. Finally, the cell pellets were placed in culture medium (0.5 ml/g liver).

Williams' Medium E was used for culture medium including the following additives: 5% fetal calf liver serum (FCS), 2 mmol/l L-glutamine, 5 ml/l human insulin (activity: 29 international units/mg). 0.8 mg/l transferrin (porcin),

3 microgramms/l glycogen (porcin), 100.000 IE/I penicillin, 100.000 microgramms/l streptomycin and 2.5 mg/l amphotericin B. The attained cell preparations were seeded into culture dishes (0.1 ml/cm² culture area), which were pre-treated for 30 minutes with a collagen solution consisting of 0.05% collagen from bovine placenta in PBS. The cultures were maintained at a temperature of 37 degrees Celsius, 5% CO₂ in air, and air humidity of 95%. After 24 hours the culture medium was replaced by fresh media (0.2 ml/cm² culture area). During the following culture phase the culture medium was replaced every 3 days. After 1-7 days single cells as well as round to oval shaped cell associations of 3-30 stem cells a1 were observed under the light microscope, which can also be referred to as colonies (FIG. 2a). Depending on the initial quality and—size of the tissue section, up to 50 such colonies were attained from one section. The cells in the colonies were predominantly of round to oval shape and, on an average, 15 micrometer in diameter; the cell nuclei covered approximately 30% of the entire cell area. In the succeeding culture process deviating cell types b2, c2 became visible, particularly in the marginal areas of the colonies. These exhibited a polygonal shape with, in part, long cytoplasmic extensions. The diameter of these cells was approximately 40 micrometer; the cell nuclei covered approximately 10% of the entire cell area. Numerous transitional cell types were also observed. The proportion of the larger polygonal cells b2 and c2 increased significantly during the culture process, whereas the proportion of the smaller oval cells decreased. To identify the cells and to evaluate their level of differentiation, the expression of specific markers for liver stem cells/liver precursor cells (CD34, c-kit, alpha-fetoprotein [AFP]), respectively differentiated hepatocytes (albumin, cytokeratin [CK] 18) and biliary epithelial cells (CK 7, CK 19) was analyzed in the cultures via indirect immune fluorescence microscopy. The tests showed the smaller, oval cells b1, c1 presented as undifferentiated/incomplete differentiated cells (CD34-, c-kit-, AFP-positive), whereas the larger polygonal cells b2, c2 located in the border areas presented as differentiated hepatocytes (albumin, cytokeratin [CK] 18 positive). Some of the slightly differentiated cells, as well as the cells located in the transitional area between differentiated and undifferentiated cells, additionally exhibited markers for biliary epithelial cells (CK 7, CK 19). For closer characterization of the cell behavior in-vitro the cultures were observed via video time-lapse microscopy over a time period of 24-96 hours. By means of the video sequences regular cell divisions was proven. C3, in FIG. 2c, specifies such cells during cell division, characterized by the large akaryote cell body, which contains the already divided chromosomes. Through immune fluorescence microscopic detection of the proliferation marker Ki-67 in the cultures, the observed mitotic activity was verified. Additionally, the video sequences also indicated that the cells marked as differentiated cells by morphological and immune fluorescence microscopical criteria, emerged from cells characterized as undifferentiated cells.

Following, procedures are described which also apply to other examples. The proliferation and differentiation behavior of the cells were influenced through variations of the applied FCS-content (0-20%), as well as through the addition of embryonal chicken extract, horse serum, growth factors, e.g. hepatocyte growth factor (HGF), transforming growth factor (TGF), insulin-like growth factor II (IGF II (2)), epidermal growth factor (EGF), stem cell factor (SCF), keratinocyte growth factor (KGF), and fibroblast growth factor (FGF), which were added separately or in combination.

Alternatively to collagen, the following extracellular matrix components were used to coat the culture dishes: fibronectin, laminin, matrigel, and heparansulfate. Co-cultures consisting of liver stem cells in combination with autologous (from the same donor) or homologous (from other donors) non-parenchyma liver cells were applied as follows: non-parenchyma liver cells were isolated through established processes. The cells were applied either untreated, or irradiated prior to application through a standard process to inhibit the proliferation of non-parenchyma cells. One approach applied a cell mixture consisting of endothelial cells, Kupffer cells, and/or Ito cells. In other applications only one non-parenchyma cell type was used. The attained cell preparations were seeded into uncoated culture dishes or into culture dishes coated with extra-cellular matrix components and maintained under standard conditions (see above). After the non-parenchyma cells had adhered and flattened, stem cell preparations were added to the cultures. Subsequently, the co-cultures were treated as described above. Co-cultures, consisting of liver stem cells in combination with autologous or homologous differentiated parenchyma liver cells, were applied as follows: differentiated parenchyma liver cells were isolated through collagenase perfusion following established processes.

In one approach the differentiated cells were mixed with stem cell preparations and seeded into culture dishes coated with extracellular matrix components. In another approach only differentiated cells were seeded. After the cells had adhered and flattened, the stem cell preparations were added to the cultures (0.1 ml/cm² culture surface). Subsequently, the cultures were treated as described above. Co-cultures consisting of liver stem cells in combination with parenchyma liver cells as well as non-parenchyma liver cells were applied as follows: first the non-parenchyma liver cells were seeded into uncoated culture dishes or culture dishes coated with extracellular matrix components. Subsequently, the differentiated cells were mixed with stem cell preparations and added to the non-parenchyma cell cultures. In another application, the individual cell fractions were seeded successively.

### EXAMPLE 2

### Extracting Liver Stem Cells from Rat Livers

To extract liver stem cells from rat livers, Wistar rats weighing 180-220 g were used. The rat was anesthetized and the liver was uncovered. Within three minutes the liver was rinsed free of blood with 20 ml PBS in situ via the portal vein and by opening of the cranial vena cava. Following, the liver was removed and the process of cell extraction was continued as described in example 1. Due to the marginal content of connective tissue in rat liver, when compared to human liver, the applied collagenase- and pronase concentrations were reduced to 0.5% and the incubation time was reduced to 30 minutes.

By reducing the used collagenase- and pronase concentrations each to 0.025% and reducing the incubation time to 15 minutes, the non-parenchyma cells remain intact to some extend. This process can be used to create co-cultures consisting of liver stem cells in combination with autologous, non-parenchyma liver cells.

### EXAMPLE 3

### Extracting Liver Stem Cells from Whole Human Organs

For the extraction of liver stem cells from whole human organs, donor organs intended for transplantation were used, which were excluded from transplantation due to damage, e.g. cirrhosis of the liver, fat liver, tic injury, or trauma. Alternatively, organs removed from transplant recipients and discarded can also be used. To fulfill the needs of the increased tissue mass of a whole human liver (approx. 1.5 kg), the method was modified.

Approach 1:

First, all blood was rinsed from the liver with a standard preservation fluid (University of Wisconsin [UW]-solution) and then stored for 48 to 72 hours at 4 degrees Celsius under exclusion of oxygen. Following, the preservation fluid was rinsed out with 31 of a PBS-solution tempered to 37 degrees Celsius, excluding calcium- and magnesium, containing 2 mmol/l EDTA. Subsequently, in order to dissolve the tissue formation, the liver was infused with 21 of enzyme solution tempered to 37 degrees Celsius, containing 0.025% collagenase type IV (activities: see example 1) in PBS without calcium- and magnesium, circulating for 20-30 minutes depending on the initial condition of the tissue.

As soon as the organ exhibited dissolution of the tissue formation, the perfusion was continued for an additional 20 minutes with 2 l of enzyme solution containing 0.1% collagenase and 0.1% pronase E (activity: see example 1) in PBS without the addition of calcium and magnesium. In the event of premature tissue breakup, the perfusion process was interrupted, and the tissue was incubated in the collagenase-pronase solution for the same amount of time at 37 degrees Celsius. After digestion, the process of cell extraction, as described in example 1, was continued. Besides the liver stem cells, non-parenchyma liver cells like endothelial cells, Kupffer cells or Ito cells remain partially intact by reducing the applied hypoxia time, collagenase- and pronase concentrations, respectively the incubation time. Therefore, this process can be used to establish co-cultures consisting of liver stem cells in combination with autologous non-parenchyma liver cells.

Approach 2:

In an alternative process the liver was preserved in a standard preservation solution (University of Wisconsin [UW]-solution) for no longer then 24 hours at 4 degrees Celsius under exclusion of oxygen. Following, the liver was digested via an established multi step perfusion process with collagenase digestion. The resulting unfractionated cell mixture, which contained the differentiated parenchyma and non-parenchyma liver cells like endothelial cells, Kupffer cells, and Ito cells as well as stem cells located in the liver was stored again for 24-48 hours at 4 degrees Celsius and under exclusion of oxygen.

Subsequently, the suspension was centrifuged at 600 RPM over 6 minutes at 4 degrees Celsius. The cell pellets were suspended in PBS and subject to one more centrifugation step (600 RPM/4° C.). Afterwards, the cells were suspended in culture medium and seeded as described in example 1. This process allows the additional extraction of differentiated parenchyma and non-parenchyma liver cells from the same organ. This can be utilized to establish co-cultures, consisting of liver stem cells in combination with autologous differentiated hepatocytes and/or autologous non-parenchyma liver cells. Hereunto, it is necessary to use a portion of the unfractionated cell mixture for the isolation of the desired cell types.

Approach 3:

The principle of this approach is to utilize the isolation process in order to simultaneously stimulate the proliferation differentiation of stem cells in vitro via simulation of natural regeneration processes. This becomes possible by generating an environment that supports stem cell proliferation, which consists of partially damaged, differentiated, parenchyma and non-parenchyma liver cells and connective tissue fragments. Signals from the perishing differentiated cells after selective damage, as well as factors released from the stem cells themselves are utilized to stimulate the proliferation of the stem cells as well as their differentiation into specific liver cell types. In this approach, the liver was digested, as described in approach 2, via an established multi step perfusion process with collagenase digestion. The attained unfractionated cell mixture, which contained the differentiated parenchyma and non-parenchyma liver cells like endothelial cells, Kupffer cells, and Ito cells as well as stem cells located in the liver, was either used for culture without additional treatment, or washed to remove debris. Thereunto, the cell mixture was centrifuged at 600 RPM for 6 minutes at 4 degrees Celsius. The cell pellets were suspended in culture medium and centrifuged again at 600 Rpm for 6 minutes at 4 degrees Celsius. Finally, 500 ml of the cell pellets were mixed with 500 ml culture medium. The cell preparation was filled into a perfuseable 3D high-density culture bioreactor with integral oxygenation and decentralized gas exchange.

This cell mixture contained the stem cells located in the liver as well as differentiated parenchyma and non-parenchyma liver cells, e.g. endothelial cells, Kupffer cells, and Ito cells. The proportion of stem cells and non-parenchyma cells was less when using centrifuged cell fractions then in the unfractionated cell mixture. In latter, the ratio of individual liver cell types to each other matched that of the liver in vivo. First the cultures were perfused for 24-96 hours under hypoxic conditions to achieve partial impairment of the differentiated parenchyma/non-parenchyma liver cells. Alternatively, a selective impairment of the differentiated cells can also be achieved through temperature changes (hypothermia, hyperthermia), chemical noxe, mechanical stress, ultrasound, pH-changes, hypotonic conditions, or other damaging factors applied individually or in combination. The cell composition, tissue organization and ultrastructure of the liver cells was immune histo-chemically and electron microscopically characterized after a 3-day to 5-week culture in 3D bioreactors.

After three to seven days of culture, large areas of perished cells were visible. In these areas, CD34- and c-kit-positive cells were regularly observed, which exhibited the morphological characteristics (size, nucleus-cytoplasm-ratio) of the liver stem cells described in example 1. Next to them, islets consisting of hepatocytes (albumin-positive) and endothelial cells (CD 31-positive) had developed, which in part, were organized into in-vivo like cell formations. Sinusoid like, anastomosing canalicular structures were also found, which were lined with endothelial cells and non-parenchyma cells (Kupffer cells, Ito cells, pit cells. In addition, several biliary duct like structures (CK 19-posiitve) had formed. Endothelial cells and biliary epithelial cells exhibited proliferation activities (Ki-67). After a 2-5 week culture process the areas with broken necrotic cells were predominantly replaced with parenchyma like tissue containing numerous, biliary duct like structures (CK 19-positive). CD-34- and c-kit-positive cells were only found sporadically.

A manipulation of the proliferation/differentiation of the cells was achieved through the variation of the applied serum content, addition of growth factors, extra cellular matrix components as well as all substances and factors mentioned in example 1.

### EXAMPLE 4

### Co-Culture of Liver Cells with Autologous Bone Marrow Cells

Theise et al. (Hepatology 2000, Bd. 31(1), S.235-240) refers to the derivation of liver precursor cells from circulating, multi-potent stem cells, which are generated in the bone marrow. The principle of the method at hand is to offer a liver specific environment to the bone marrow stem cells to induce the differentiation of the cells towards liver cells.

Human bone marrow stem cells were extracted from the sternum or vertebrae aspirate of organ donors whose livers were not suitable for transplantation because of damage, liver cirrhosis, fatty liver, or traumatic impact. The aspirates were mixed with 1% heparin and 10% Terasaki-Park-medium. Mononuclear cells were attained through density gradient centrifugation with Ficoll separation solution (density 1.077) followed by two centrifugation steps in Hanks' balanced salt solution (HBSS) to wash the cells. Following, the cells were suspended in Terasaki Park Medium with 0.5% FCS. By way of cytocentrifugation, the cells were placed on a microscopy slide for morphological characterization and stained according to Pappenheim. The clonogenity of the mononuclear cells was tested through clonogenic assays (MethoCuit™ GF H4434, StemCell Technologies). The mononuclear cells were mixed with autologous liver cells, which were extracted through one of the processes described in example 3, at a 1:10 to 1:100 ratio and suspended in basal ISCOVE-Medium, which contained 20% heat activated FCS, 1 mmol/l natrium pyruvate, 2 mmol/l L-glutamine, 100,000 IE/l penicillin, 100,000 micrograms/I streptomycin and 2.5 mg/l amphotericin B.

Approach 1:

The cell preparations were seeded into culture dishes (0.1 ml/cm² culture area, which had been treated for 30 minutes with a collagen solution consisting of 0.05% collagen from cattle placenta in PBS prior to use. For the identification of the cells and the evaluation of their level of differentiation, the expression of specific, common markers for bone marrow stem cells and liver stem cells (CD34, Thy-1) as well as liver precursor cells (AFP), respectively differentiated hepatocytes (albumin, CK 18) and biliary epithelial cells (CK 7, CK 19), was investigated in the cultures through indirect immune fluorescence microscopy. Cells/colonies that were only CD34- and Thy-1-positive, as well as colonies that additionally expressed AFP and CK 18 and 19, and some that exhibited markers for differentiated liver cells were detected. In comparison to the described liver cell monocultures, the number of the resulting colonies containing undifferentiated liver cells was significantly higher.

Approach 2:

The bone marrow stem cells were co-cultured in bioreactors with autologous liver cells (see example 2, approach 3). For this purpose, the bone marrow stem cells were mixed with liver stem cell preparations/unfractionated liver cell mixtures at a ratio of 1:10 to 1:100 and then inoculated into bioreactors. In a further approach first the liver cells were inoculated and only after the liver cells reorganized inside the bioreactor, the bone marrow stem cells were added to the cultures. Some bioreactor cultures were perfused under hypoxic conditions for 24-96 hours to achieve a partial impairment of the differentiated parenchyma/non-parenchyma liver cells. The cell composition, tissue organization and ultra structure of the cells after 3-day to 5-week culture in a 3D-bioreactor was characterized by immune histochemistry and electron microscopically characterized.

After a 3 to 10 day culture process numerous islets consisting of bone marrow stem cells (CD34-positiv), surrounded by parenchyma and non-parenchyma liver cells were observed. In some islets, liver precursor cells (CD34- and AFP-positive) were also observed, which exhibited morphological characteristics (size, nucleus-cytoplasma-ratio) of the liver stem cells described in example 1. Alongside, isles with hepatocytes (albumin-positive) and endothelial cells (CD31-positive) had formed, which were arranged into in-vivo like cell formations as described in example 3, approach 3. After a 3 to 5 week culture process, the bioreactors contained primarily parenchyma like tissue. Bone marrow stem cells were only seen sporadically.

In further approaches, the bone marrow stem cells were co-cultivated with autologous bone marrow stroma cells and/or non-parenchyma liver cells (see example 1). A manipulation of the cell proliferation/differentiation could also be achieved through variation of serum content, addition of growth factors, extracellular matrix-components, as well as all substances and factors described in example 1.

1. Method to manufacture cell preparations thereby characterized that an unfractionated cell mixture of an organ or tissue is generated and the cells with a predetermined degree of maturity and/or differentiation are selectively and/or partially damaged.

2. Method according to above described claim thereby characterized that the damaging of the matured, highly differentiated cells occurs before, simultaneously with, or after the generation of the unfractionated cell mixture.

3. Method according to one of afore described claims thereby characterized that cells with a pre-determined degree of impairment and/or a predetermined degree of maturation and/or—differentiation are extracted from the cell preparation.

4. Method according to one of afore described claims thereby characterized that the cell preparation is maintained under culture conditions for proliferation, differentiation, and/or maintenance.

5. Method according to one of afore described claims thereby characterized that the cells are taken into co-culture with other differentiated cells of the same and/or another defined organ- and/or tissue type (of an organ and/or tissue) of the same and/or another organism before, simultaneously with, or subsequent to selective and/or partial damage.

6. Method for the production of a cell preparation thereby characterized that one or several stem cells are cultivated in co-culture with other differentiated cells of a pre-determined organ and/or tissue.

7. Method according to one of afore described claims thereby characterized that the stem cells are maintained in co-culture under culture conditions with other differentiated cells of the same and/or another pre-determined organ, and/or pre-determined tissue type (of an organ, and/or tissue) of the same and/or another organism for proliferation, differentiation, and/or maintenance.

8. Method according to claims 5 to 7 thereby characterized that the other differentiated cells are selectively and/or partially damaged before and/or after generating the co-culture.

9. Method according to claims 5 to 8 thereby characterized that the stem cells are maintained separately from the other differentiated cells through a barrier not permeable for cells.

10. Method according to afore claim thereby characterized that a barrier is used that is permeable or semi-permeable for active agents, mediators, and/or metabolic products of cells.

11. Method according to one of the two afore described claims thereby characterized that a membrane not permeable for cells, for example a hollow fiber or flat membrane, is used as barrier.

12. Method according to above described claim thereby characterized that a membrane is used that exhibits pores which facilitate cell-cell communication.

13. Method according to one of afore described claims thereby characterized that following the selective and/or partial damage of the cells, non-parenchymal and/or mesenchymal cells of the same and/or another organ and/or tissue type of the same and/or another organism are added.

14. Method according to one of afore described claims thereby characterized that bio-matrix proteins, e.g. collagens or fibronectin, are added to the cell preparation.

15. Method according to one of afore described claims thereby characterized that the cell preparation is cultivated before, simultaneously with, and/or after the selective and/or partial damage in an organ- and/or tissue specific environment.

16. Method according to one of afore described claims thereby characterized that the cell preparation is cultivated in a module. The module consists of an outer casing, and at least three independent membrane systems, whereby at least two independent membrane systems are designed as hollow fiber membranes and are arranged in the interior of the module. These hollow fiber membranes form a tightly packed network. The cells are arranged inside the hollow spaces of the network and/or adhere to the hollow fiber membranes (3). This network, consisting of intersecting and/or overlaying hollow fiber membranes, is constructed in such a way that the cells have almost identical substrate supply and removal conditions from anywhere inside the module (1).

17. Method according to claim 16 thereby characterized that the tightly packed network in the interior is formed through three independent hollow fiber membrane systems.

18. Method according to claims 16 or 17 thereby characterized that an interchangeable flat membranes or capillary membranes are additionally affixed to the outer casing.

19. Method according to claims 16 to 18 thereby characterized that the tightly packed network also exhibits an additional fluid impermeable independent capillary system.

20. Method according to claims 16 to 19 thereby characterized that the outer casing is generated from a casting whereby an inlet facilitates access from the outside into the lumen of the capillaries or hollow fiber membranes.

21. Method according to claims 16 to 20 thereby characterized that in- and/or outlet heads (6, 13, 14, 15) are provided that communicate with the respective independent capillary system to facilitate the inlet into and/or outlet from the lumen of the capillaries or hollow fiber membranes.

22. Method according to claims 16 to 21 thereby characterized that the casing of the module is equipped with one or more accesses into the interior to fill microorganisms into the module and/or conduct pressure-, temperature, and/or pH-measurements.

23. Method according to claim 22 thereby characterized that the accesses ways continue into the module as perforated tubes, which facilitates an even distribution of the microorganisms in the interior.

24. Method according to one of afore described claims thereby characterized that the cell preparation is cultivated inside a module, consisting of a body made of porous material whose pores communicate with each other, and at least one channel like hollow pathway system whose hollow pathways intersect and/or overlay each other, penetrate the body, and is arranged inside a water-/germ tight container.

25. Method according to claim 24 thereby characterized that it exhibits at least two independent channel like hollow pathway systems.

26. Method according to claim 25 thereby characterized that one channel like hollow pathway system consists of parallel running individual channels arranged in at least one plane.

27. Method according to claim 26 thereby characterized that a hollow pathway system is formed of several planes arranged on top of each other and each consists of parallel arranged individual channels.

28. Method according to one of the claims 25 to 27 thereby characterized that three independent channel like hollow pathway systems are present.

29. Method according to one of the claims 25 to 28 thereby characterized that four independent hollow pathway systems are present.

30. Method according to at least one of the claims 25 to 29 thereby characterized that the diameter of each individual channel of the channel like hollow pathway systems is 0.1-2 mm.

31. Method according to at least one of the claims 25 to 30 thereby characterized that the spacing between the individual, parallel running channels of a hollow pathway system, arranged in one plane, and/or in between a plane is 1-5 mm.

32. Method according to at least one of the claims 25 to 31 thereby characterized that the pores of the body are 100-1000 micrometer in diameter.

33. Method according to at least one of the claims 25 to 32 thereby characterized that the pores are interconnected through hollow spaces of 50-300 micrometer in size.

34. Method according to at least one of the claims 25 to 33 thereby characterized that the body is a formation of several, each other overlaying, individual, disc/slide like layers that are held together by the container.

35. Method according to at least one of the claims 25 to 34 thereby characterized that the disc/slide like individual layers are penetrate, in at least one layer, with channel like ridges that are arranged and dimensioned in such a way that they form a channel like hollow pathway system in connection with the next following individual layer.

36. Method according to claim 34 or 35 thereby characterized that the front wall of the disc/slide like individual layers are penetrated with a channel like hollow pathway system.

37. Method according to claim 36 thereby characterized that the disc/slide like individual layers are penetrated with hollow pathways from one plane to the next.

38. Method according to one of the claims 25 to 37 thereby characterized that the channel like hollow pathways of a system meet in at least one inlet and one outlet.

39. Method according to claim 38 thereby characterized that the inlet and outlet is connected to the porous body.

40. Method according to claim 39 thereby characterized that the inlet and outlet is an integral part of the body.

41. Method according to claim 40 thereby characterized that the porous material consists of a sintered ceramic powder material.

42. Method according to one of the afore described claims thereby characterized that the cell preparation is cultivated in at least one bioreactor in the form of a perfuseable organ copy consisting of an immunological inactive porous body, whose pores communicate with each other, and organ specific hollow structures.

43. Method according to claim 42 thereby characterized that the pores of the bioreactor are 50-1000 micrometer in diameter.

44. Method according to claims 42 or 43 thereby characterized that the pores of the bioreactor are 50-1000 micrometer in diameter.

45. Method according to one of the claims 42-44 thereby characterized that the organ copy is arranged in a liquid- and germ tight container and that the outer casing is equipped with connections that are connected with at least one hollow structure of the organ copy.

46. Method according to at least one of the claims 42-45 thereby characterized that the container and the connections consists of a biodegradable material.

47. Method according to at least one of the claims 42-43 thereby characterized that the porous body consists of a biodegradable material.

48. Method according to one of the claims 42-43 thereby characterized that the porous body consists of a sintered ceramic powder.

49. Method according to one of the claims 42-46 thereby characterized that it is a copy of the liver, bone marrow, lymph nodes, thymus, spleen, kidney, pancreas, pancreatic islet organ, mucosa membrane, thyroid gland, parathyroid gland, adrenal gland, bone, gonads, uterus, placenta, ovaries, blood vessels, heart, lungs, muscle, intestinal wall, bladder, heart muscle, brain, neural tissue, and/or other mammalian organs.

50. Method according to one of afore described claims thereby characterized that the stem cell preparation is stored cooled or frozen.

51. Method according to one of afore described claims thereby characterized that before and/or after selective and/or partial damage, the regeneration, proliferation and/or differentiation of the cells is stimulated through the addition of active agents, growth factors, and/or differentiation factors.

52. Cell preparation containing an unfractionated cell mixture of an organ and/or tissue thereby characterized that the cells with a predetermined degree of maturity and/or differentiation are selectively and/or partially damaged.

53. Cell preparation according to afore described claim thereby characterized that the cells were damaged before, simultaneously with, or after the generation of the unfractionated cell mixture.

54. Cell preparation according to one of the two afore described claims thereby characterized that the cells with a predetermined degree of damage and/or a predetermined degree of maturity and/or degree of differentiation are separated and extracted from the cell preparation.

55. Cell preparation according to one of the claims 52 to 54 thereby characterized that it contains other additional differentiated cells of the same and/or another specific organ- and/or tissue type (of an organ and/or tissue) of the same and/or another organism.

56. Cell preparation containing a culture of stem cells thereby characterized that it contains additional differentiated cells of a predetermined organ and/or tissue.

57. Cell preparation according to afore claim thereby characterized that it contains other additional differentiated cells of the same and/or another specific organ and/or tissue type of an organ and/or tissue of the same and/or another organism.

58. Cell preparation according to claim 56 or 57 thereby characterized that it generates therapeutically usable, organ regenerating factors.

59. Cell preparation according to one of the claims 55 to 58 thereby characterized that the other differentiated cells are selectively and/or partially damaged

60. Cell preparation according to one of the claims 55 to 59 thereby characterized that the cells are separated from the other differentiated cells through a barrier not permeable for cells.

61. Cell preparation according to afore described claim thereby characterized that the barrier is permeable or semi-permeable for active agents and/or metabolic products of cells.

62. Cell preparation according to one of the two afore described claims thereby characterized that the barrier is a membrane not permeable for cells.

63. Cell preparation according to one of the claims 52 to 62 thereby characterized that it contains non-parenchymal cells and/or mesenchymal cells of the same and/or another organ- and/or tissue type of the same and/or another organism.

64. Cell preparation according to one of the claims 52 to 63 thereby characterized that it contains biomatrix proteins, for instance collagen or fibronectin.

65. Cell preparation according to one of the claims 52 to 64 thereby characterized that it is arranged in an organ- and/or tissue specific environment.

66. Cell preparation according to one of the claims 52 to 65 thereby characterized that the cell preparation is cultivated inside a module that exhibits an outer casing and at least three independent membrane systems, whereby at least two independent membrane systems are formed as hollow fiber membranes, which form a tightly packed network containing of intersecting and/or each other overlaying hollow fiber membranes.

67. Cell preparation according to claim 65 thereby characterized that the cell preparation is cultivated inside a module that exhibits a porous body contained inside an outer casing. The pores of this porous body communicate with each other and it contains at least two independent channel systems that interconnect and/or over laying each other and penetrate the body.

68. Cell preparation according to one of the claims 52 to 67 thereby characterized that they are stored cooled or frozen.

69. Cell preparation according to one of the claims 52 to 68 thereby characterized that it contains substances for the regeneration, proliferation, differentiation and/or maintenance of cells.

70. Cell preparation thereby characterized that it is producible or was produced according to one of the claims 1 through 51.

71. Utilization of a process or a cell preparation according to one of afore described claims for the production of a stem cell culture, a progenitor cell culture, or a stem cell culture of a mammal or a human.

72. Utilization according to afore described claim for the generation of a culture of somatic stem cells.

73. Utilization according to one of the two afore described claims for the generation of a stem cell culture of an organ and/or tissue.

74. Utilization according to afore described claim for the generation of an organ specific stem cell culture from liver, bone marrow, lymph nodes, thymus, spleen, kidney, pancreas, pancreatic islet organ, mucosa membrane, thyroid gland, parathyroid gland, adrenal gland, bone, gonads, uterus, placenta, ovaries, blood vessels, heart, lungs, muscle, intestinal wall, bladder, heart muscle, brain, neural tissue, and/or other mammalian organs.

75. Utilization of a process or a cell preparation according to one of the claims 1 through 70 for the examination of the effect of the metabolism and/or toxicity of chemicals and/or pharmaceuticals and/or for the development of pharmaceuticals.

76. Utilization of a process or cell preparation according to one of the claims 1 through 70 for the extraction of substances that stimulate and/or support the proliferation and/or differentiation of stem cells in vivo and/or in vitro, especially differentiation factors, reproductive factors, growth factors, mediators, cytokines and/or hormones.

77. Utilization of a process or cell preparation according to one of the claims 1 through 70 as cell implant/modified transplant or for extracorporeal organ-/tissue replacement.

78. Utilization of a process or cell preparation according to one of the claims 1 through 70 for the extraction of substances that stimulate the regeneration of diseased organs and can be applied locally, orally, or systematically in regenerative medicine.

79. Utilization of a process or cell preparation according to one of the claims 1 through 70 for the production of connective tissue-/stroma cells for the Feeder-layer-culture and stem cell co-culture.

80. Utilization of a process or cell preparation according to one of the claims 1 through 70 for in vitro virus replication systems as well as vaccine production.

81. Utilization of a process or cell preparation according to one of the claims 1 through 70 for the production of hybrid hematopoietic bone marrow.

82. Utilization of a process or cell preparation according to one of the claims 1 through 70 for the production of a hybrid immune system for the generation of immune competent cells, antibodies, or vaccines.

83. Method, according to afore described claims, for the production of an autologous organ transplant for transplantation into a patient thereby characterized that a homologous organ transplant is infused with the patient's own cells, and the original cells of the organ transplant are selectively and/or partially damaged.

84. Method according to afore described claim thereby characterized that the patient's own cells consist of an unfractionated cell mixture.

85. Method according to afore described claims thereby characterized that the original material stems from fetal cells, including human fetal cells.

86. Method according to afore described claims thereby characterized that the original material stems from stem cell lines or embryonal stem cells, including human embryonal cells.
